(19) United States
(12) Patent Application Publication
(10) Pub. No.: US 2017/0020723 A1
(43) Pub. Date: Jan. 26, 2017

(54) PATIENT COMFORT PACK

(71) Applicant: Sara Anne Szokola, Plymouth, MI (US)

(72) Inventor: Sara Anne Szokola, Plymouth, MI (US)

(21) Appl. No.: 15/052,871

(22) Filed: Feb. 24, 2016

Related U.S. Application Data
(60) Provisional application No. 62/119,896, filed on Feb. 24, 2015.

(51) Int. Cl. A61F 7/10; A61F 7/08

(52) U.S. Cl. CPC A61F 7/106; A61F 7/08; A61F 2007/0041

(57) ABSTRACT

The present invention provides a device and method for delivering thermal therapy about a percutaneous access site while also permitting visual and physical examination of the access site. The device comprises a semi-circumferential shaped housing having a top wall, a bottom wall, and a side wall. The top wall, the bottom wall, and the side wall define a cavity with a fluid tight bladder disposed within the cavity. The semi-circumferential shaped housing has an aperture formed in the housing and a straight edge portion along the circumference of the housing. The device delivers thermal therapy to percutaneous arterial, venous, laparoscopic, and other access procedures sites by allowing visual and physical examination of the access site through the aperture and comfortable placement of the device's straight edge portion against a bend or joint of the patient's body.

### CROSS-REFERENCE TO RELATED APPLICATION

The present application claims priority to U.S. Provisional Patent Application No. 62/119,896, filed on Feb. 24, 2015, entitled “Patient Comfort Pack” the entire disclosure of which is incorporated by reference herein.

### BACKGROUND OF THE INVENTION

1. Field of invention

The present invention relates generally to thermal packs and more particularly to an external, sterile anti-inflammatory thermal pack for placing comfortably about an access site to minimizing swelling, pain and discomfort.

2. Description of Related Art

Commercially available thermal packs are widely known and useful for providing heat or cold therapy for a variety of rehabilitative purposes such as pain relief, reduction of inflammation and edema. The heat or cold therapy is typically delivered by thermal packs that are capable of delivering or absorbing heat through the use of compounds that may be heated or cooled manually or create heat or cooling through instantaneous chemical reactions such as with instant heat or cold packs. Unfortunately, the currently available thermal packs are not optimally designed for comfortable placement about a percutaneous access site while also permitting visual and physical examination of the access site.

Today, physicians gain access to vascular anatomy by puncturing an artery, such as the femoral, radial, brachial, pedal and popliteal arteries, large enough to allow access for a sheath, wire, guide, or interventional device designed to treat a wide array of atherosclerosis. Upon completion of the procedure, it is common for the physician to use an arterial closure device. This closure device is a synthetic dissolving plug that seals the artery and stops external bleeding (hemostasis). Once hemostasis has been achieved it is common for the physician or a staff member to place a piece of sterile gauze over the access site. Over the next several hours, the physician and hospital staff will monitor the patient closure site. This may include a visual inspection and/or physical examination around the access site area to ensure there are no complications such as hematoma, external bleeding, extreme discomfort, tenderness, and/or swelling.

Another procedure frequently used by general surgeons, obstetricians and gynecologists, and urologists is the use of laparoscopic ports to perform minimally invasive surgery. The use of this technique has expanded greatly over the last ten (10) years and today thousands of laparoscopic procedures are performed on a daily basis. Laparoscopic surgery uses more than one 0.5-1.0 cm incision. Each incision is called a “port.” At each port a tubular instrument known as a trochar is inserted. Specialized instruments and a special camera known as a laparoscope are passed through the trochars during the procedure. At the end of the procedure it is common for the physician to close the access ports with a needle and bio-absorbable suture. As with other closure sites, a visual inspection or examination of the access site area is performed to ensure there are no complications and determine the existence of a hematoma, external bleeding, extreme discomfort, tenderness and/or swelling.

The access sites created by laparoscopic procedures, arterial access, and the like result in damage to muscle and connective tissue thereby triggering a number of physiological processes. For example, the area may bleed depending on the severity of the damage. The bleeding and damage to the tissue set off a cascade of chemical reactions, with the end result of edema (swelling). As the area swells, the blood and oxygen supply to the surrounding tissues becomes compromised. At this point, undamaged cells in the immediate vicinity of the damaged muscle and connective tissue become vulnerable to a process called secondary hypoxic injury. Thus, the undamaged cells may begin to die from a lack of oxygen due to the compromised blood supply to the area surrounding the access site.

Disposable or reusable instant cold packs are often used to provide post-operative rehabilitation. U.S. Pat. No. 6,916,334 and U.S. Pat. No. 5,679,052 describe thermal packs for delivering therapy to the breast by providing a donut-shaped or c-shaped device accommodating the nipple and containing a gel capable of delivering heat or cold therapy. The donut-shaped or c-shaped devices, however, are limited in their application to areas of the body where such a shape can fit comfortably such as the breast.

A need therefore exists for a thermal pack configured to fit comfortably in the region of arterial or laparoscopic access while also permitting physicians and medical staff the ability to monitor the access site for post procedural complications.

### SUMMARY OF THE INVENTION

The present invention overcomes these and other deficiencies of the prior art by providing a therapeutic thermal device for application about an access site. The therapeutic thermal device comprises a semi-circumferential shaped housing having a top wall, a bottom wall, and a side wall. The top wall, the bottom wall, and the side wall define a cavity that includes a fluid tight bladder disposed within the cavity. The semi-circumferential shaped housing has an aperture formed in the housing and a substantially rectangular portion along the circumference of the housing. The substantially rectangular portion is configured to allow for the comfortable placement of the device in the region of arterial or laparoscopic access. The aperture formed in the housing allows physicians and medical staff to monitor the access site for post procedural complications.

The present invention is a therapeutic thermal device that may be used for cryotherapy or heat therapy. Accordingly, also disclosed is a method for application of a therapeutic thermal device. The aperture formed in a semi-circumferential housing of the device having a top wall, a bottom wall, and a side wall, the top wall, the bottom wall, and the side wall defining a cavity is placed over an access site. The placement of the aperture over the access site allows for orienting a straight edge portion along the circumference of the semi-circumferential housing at a bend or joint on a body located adjacent to an access site such as the location of the femoral, radial, brachial, pedal and popliteal arteries. Cryotherapy or heat therapy may then be applied directly to the area surrounding the access site or indirectly to the access site from a chemical reaction occurring in a fluid tight bladder disposed within the cavity defined by the top wall, the bottom wall, and the side wall, which includes a solution and a separately maintained compartment for an organic or inorganic salt to provide thermal therapy.

The present invention is designed to fit comfortably in the region of arterial or laparoscopic access. Because arterial and laparoscopic access sites are adjacent to bends or joints of the limbs of the human body, comfortable placement of a therapeutic thermal pack is difficult. In addition, post-procedure, the arterial and laparoscopic access sites may experience procedural complications requiring medical observation and physical examination. The present invention addresses this need by providing a therapeutic thermal pack configured to be comfortably placed in the region of arterial or laparoscopic access. The present invention includes an aperture in the device for visual and physical examination of the access site while being comfortably placed in the region of arterial or laparoscopic access.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, and the advantage thereof, reference is now made to the ensuing descriptions taken in connection with the accompanying drawings briefly described as follows:

FIG. 1 illustrates a top view of the device according to an embodiment of the invention; and

FIG. 2 illustrates an alternative top view of the device according to an embodiment of the invention.

### DETAILED DESCRIPTION OF EMBODIMENTS

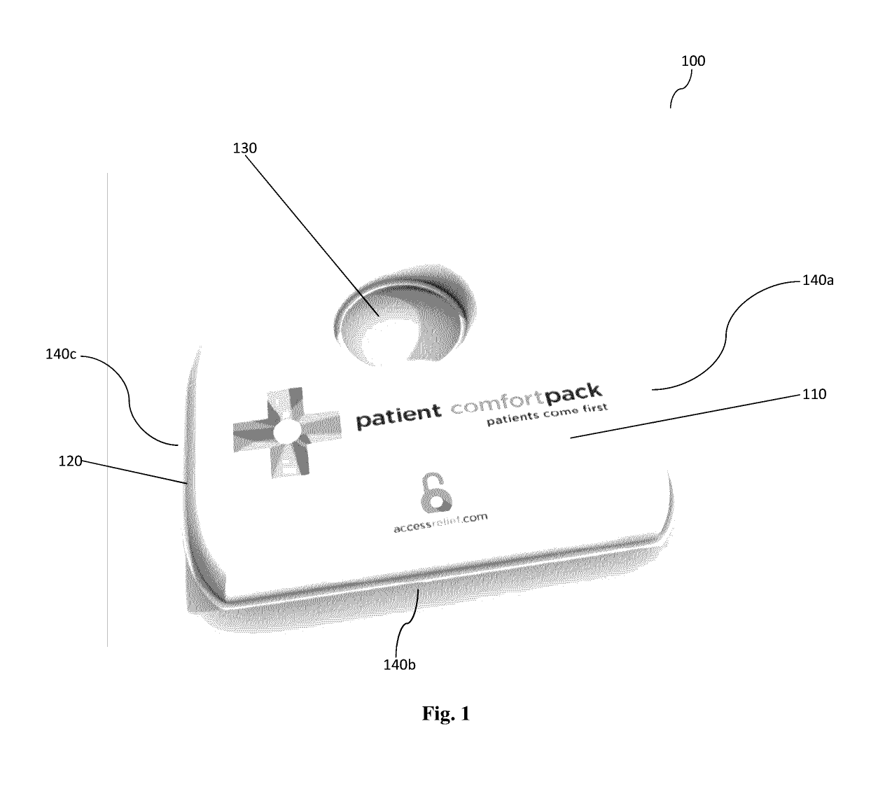

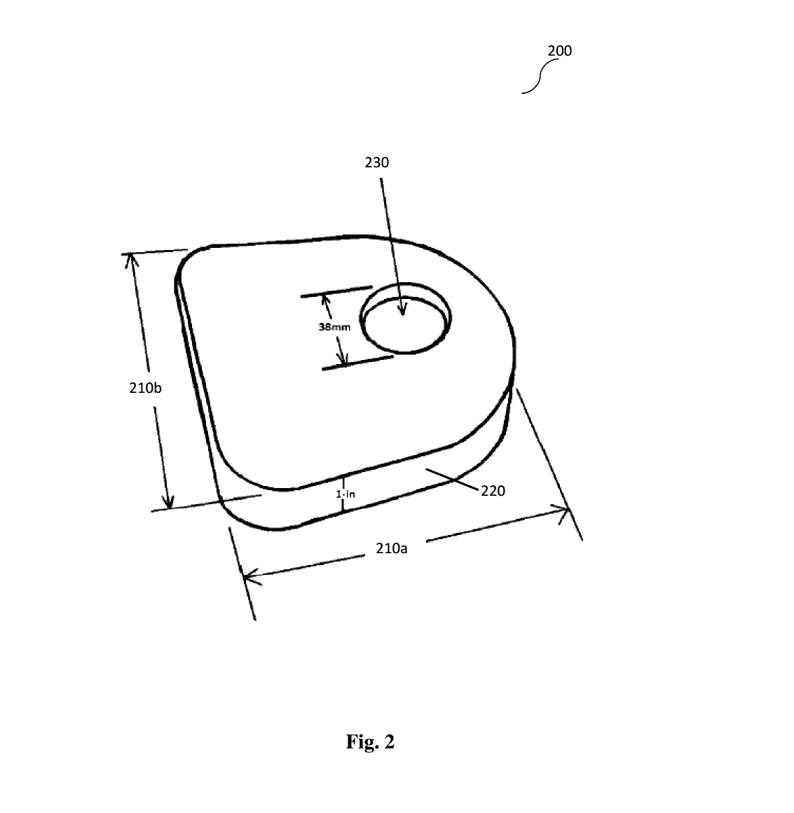

Further features and advantages of the invention, as well as the structure and operation of various embodiments of the invention, are described in detail below with reference to the accompanying FIGS. 1-2. Although the invention is described in the context of a cold pack, it is to be understood that the present invention is equally capable of delivering heat therapy to an access site. One of ordinary skill in the art will understand the implementation of therapeutic heat delivery via an exothermic reaction within the device of the present invention.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Moreover, the described features, structures, or characteristics of the invention may be combined in any suitable manner in one or more embodiments. It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents. Reference will now be made in detail to the preferred embodiments of the invention.

Currently, there are no external, sterile anti-inflammatory medical cold packs capable of treating percutaneous access sites at the femoral, radial, brachial, pedal and popliteal arteries or laparoscopic port access sites after the site has been closed. In addition, there are no cold packs on the market specifically designed to treat the surrounding damaged tissue at the access site while allowing the physician and staff the ability to palpate and visualize the access wound site. The area at and surrounding the access site frequently experiences post procedural complications and post operative pain. For example, these complications may include access site swelling, discomfort, bruising, inflammation and hematoma. Today, there are no sterile cold packs on the market specifically designed to address external arterial access site complications such as hematoma, discomfort, tenderness and swelling and also capable of comfortable placement about the access site.

The device design and mechanism of the present invention minimizes swelling, pain and discomfort resulting at the access site after a procedure such as laparoscopy or arterial access. In an embodiment of the invention, a sterile semi-circumferential therapeutic cold pack is placed over an access closure site. In an arterial access procedure, the access site may be located at the femoral, radial, brachial, pedal or popliteal artery. The present invention allows the surrounding tissue at the access site to receive direct cryotherapy slowing the inflammatory response and reducing pain. The sterile semi-circumferential therapeutic cold pack includes an aperture. Because the aperture is surrounded by the sterile semi-circumferential therapeutic cold pack, the access site and immediately surrounding tissue also receive indirect cryotherapy. The aperture also allows attending physicians and medical staff to palpitate and visually examine the access site for post procedural complications and post operative pain. As such, placement of the sterile semi-circumferential therapeutic cold pack should be such that the access site is within the aperture to provide visualization, palpitation and indirect cryotherapy.

In an embodiment, the device may be used at an access site after hemostasis is achieved by either an access closure device or manual hold. The device of the present invention is designed to provide both direct and indirect cyotherapy to an area adjacent to and at an access site. In some embodiments, the direct cryotherapy area extends at least 3.5 cm radially from the access site. In alternative embodiments, the direct cryotherapy area extends radially from the access site a distance sufficient to provide direct cryotherapy to an area known to one of skill in. the art that may be exposed to secondary hypoxic injury and/or other post-procedural complications. The direct cryotherapy area is the area where the device of the present invention is in direct contact with the skin. The device may be made of a flexible rubber/silicone outer shell or the like. The outer shell may be a latex-free thin film based medical grade polymer such as a thermoplastic elastomer copolyester, polyvinyl chloride, or the like.

In a preferred embodiment, the device is in a semi-circumferential shape. By semi-circumferential, it is meant and intended that the device have a shape as shown in the non-limiting examples of FIGS. 1 and 2. The upper portion of the device is semi-circumferential with the lower portion of the device configured such that there are three substantially straight edges forming three sides of a substantially rectangular shape. An aperture is formed in the upper portion of the device allowing physicians and medical staff to palpitate and visually examine the access site for post procedural complications and post operative pain. Accordingly, the aperture is located in the upper portion of the device to facilitate placement of the aperture directly over the access site. Orientation with the aperture directly over the access site allows for the comfortable placement of the straight edge portions of the device against the groin or other bend or joint adjacent to arterial access sites such as located at the femoral, radial, brachial, pedal or popliteal artery.

In an alternative embodiment as illustrated in the figures of U.S. Provisional Patent Application No. 62/119,896 incorporated herein by this reference, the device includes a singular straight edge portion along the circumference of the semi-circumferential shape. In this alternative embodiment, the length of the singular straight edge portion of the device is approximately one-quarter (25%) of the circumference of a complete circle with a given diameter. For example, if the device was a complete circle with a diameter of 10 cm, then the circumference of the circle would be 31.4 cm (c=πd, where c=circumference and d=diameter of a circle). One-quarter of the circumference would then be 7.9 cm. Therefore, the straight edge would have a length of approximately 8 cm. In alternative embodiments, the length of the straight edge portion is between 5% and 30% of the circumference of the device if the device was a complete circle. The straight edge portion facilitates placement of the device adjacent to a patient's inner groin or any location on the body where a bend or joint may be located adjacent to an access site such as the location of the femoral, radial, brachial, pedal and popliteal arteries.

FIG. 1 illustrates a top view of the device 100 according to an embodiment of the present invention. The outer shell of the device includes a top wall 110, a side wall 120, and a bottom wall (not shown) forming a fluid digit cavity. An aperture 130, located in the upper portion of the device 100 of the semi-circumferential shape, exposes the access site for visual and physical examination while also allowing for indirect cryotherapy. The lower portion of the device 100 includes three substantially straight edges, 140a, 140b, and 140c, continuous with the semi-circumferential upper portion of the device. The three substantially straight edges, 140a, 140b, and 140c, allow for the comfortable placement of the therapeutic thermal device against the inner groin or any location on the body where a bend or joint may be located adjacent to an access site such as the location of the femoral, radial, brachial, pedal and popliteal arteries. The three substantially straight edges, 140a, 140b, and 140c, combine to form a substantially rectangular shape defining the bottom portion of the device 100. The straight edges, 140a and 140c, are at a slight tapered configuration to allow for correct and comfortable placement against the inner groin. This slight tapered configuration aligns with the angle formed in the groin region where the leg meets the groin. In other embodiments, the straight edges, 140a, 140b, and 140c, may be configured to adapt to the area adjacent to an access site such as at the bend or joint at the location of the femoral, radial, brachial, pedal and popliteal arteries. Once the access site is placed within the aperture 130, the device may be correctly oriented for comfortable placement against a bend or joint adjacent to an access site.

FIG. 2 illustrates an alternative top view of the device 200 according to an embodiment of the invention. In a preferred embodiment, the dimensions of the device 200 are equal along the periphery of the device as shown in FIGS. 2 at 210a and 210b. The specific lengths of the periphery of the device are configured to accommodate the specific end use of the device. For example, the dimensions will be dictated by the location of the access site i.e., at the femoral, radial, brachial, pedal and popliteal arteries. Moreover, the dimensions of the device are dependent upon the thermal qualities of the reactive chemicals used in the device to produce heat or cold therapy. In this preferred embodiment. the side wall 220 of the device is at least 1 inch in height. It is to be understood that the specific height of the side wall 220 may vary depending on the thermal qualities of the reactive chemicals used in the device to produce heat or cold therapy. For example, the height of the side wall 220 may vary depending upon the choice of reactive chemicals used to produce endothermic or exothermic reactions. The choice of solution and dry chemicals may dictate the specific amounts needed to achieve an appropriate heat of reaction (ΔH) necessary to produce the desired therapeutic thermal effect. In this preferred embodiment, the aperture 230 has a diameter of at least 38 millimeters to allow visual and physical examination of the access site. It is to be understood that the diameter of the aperture 230 will vary dependent upon the end use of the device. For example, if the end use of the device is for use with an access site that requires a larger or smaller viewing and examination area, then the aperture may be larger or smaller to accommodate that particular end use.

The bottom wall (not shown) is identical to the top wall 110 of FIG. 1 but includes at least one sterile-unitary peel away paper that exposes at least one adhesive point. The at least one sterile-unitary peel away paper exposes at least one adhesive point. The at least one adhesive point temporarily adheres the device to the skin such that the aperture exposes the access site and the bottom wall rests on the area adjacent to the access site. The adhesives used in the device may be any adhesive appropriate for contacting a patient's skin known to one of skill in the art. In an embodiment of the device, the adhesive is a medical grade adhesive glue similar to self sticking bandages. The adhesive may be a breathable polymer based pressure sensitive adhesive including a slight liquid carrier that will facilitate bonding with a patient's skin.

The device as described herein is a cryotherapy device capable of absorbing heat. As such, the device includes a fluid tight inner bladder (not shown) disposed between the top wall 110 and the bottom wall (not shown) of FIG. 1. In an embodiment of the invention, the inner bladder includes water and either an organic or inorganic salt maintained separately from the water. To initiate an endothermic reaction, the salt is dissolved in the water. This process is known in the art and used in many commercially available instant cold packs. Typically, dry chemicals are maintained in a small bag within the inner bladder containing an aqueous solution. The small bag is manually manipulated or crushed thereby causing the dry chemicals to be released from the bag and dissolved in the surrounding solution. The salts used in the reaction may be any salt, including but not limited to ammonium nitrate, calcium ammonium nitrate, potassium nitrate, urea, ammonium chloride, or the like. It is to be understood that the particular salt and solution used in the endothermic reaction have a heat of reaction (ΔH) known to those of skill in the art capable of providing cryotherapy to a patient in a post procedure setting.

In an embodiment of the invention, the placement of the aperture in the device is off center such that the inner bladder is of a sufficient size to accommodate an endothermic reaction with a heat of reaction capable of providing at least 25-30 minutes of cryotherapy. In alternative embodiments, the aperture in the device may be oriented at a location on the device dependent on the size of the inner bladder required to produce an endothermic reaction with a heat of reaction capable of providing cryotherapy for a length of time given the particular end use of the therapy. For example, cryotherapy for lengths of time greater or less than 30 minutes would require an inner bladder that is larger or smaller to accommodate the chemicals needed to create an endothermic reaction for that particular period of time.

In alternative embodiments, the device is a heat therapy device capable of delivering heat to an area at an access site. Those of skill in the art understand the implementation of this embodiment within the device of the present invention. For example, a supersaturated solution of sodium acetate may be used whereby activation of the exothermic reaction occurs by creating a nucleation site for the crystallization of sodium acetate thereby releasing heat.

The specific sterile design of the present invention addresses patient and physician requests to minimize swelling from hematoma, discomfort, tenderness and swelling at an arterial access site, while still allowing for visualization and palpitation. The device addresses multi-physician specialties, while satisfying an unmet patient need. Percutaneous arterial, venous, laparoscopic, and other access procedures are addressed through the use of this device.

The above description of the disclosed embodiments, and that provided in the accompanying documents, is provided to enable any person skilled in the art to make or use the invention. Various modifications to these embodiments will be readily apparent to those skilled in the art, and the generic principles described herein, and in the accompanying documents, can be applied to other embodiments without departing from the spirit or scope of the invention. Thus, it is to be understood that the description and drawings presented herein, and presented in the accompanying documents, represent particular aspects and embodiments of the invention and are therefore representative examples of the subject matter that is broadly contemplated by the present invention. It is further understood that the scope of the present invention fully encompasses other embodiments that are, or may become, obvious to those skilled in the art and that the scope of the present invention is accordingly not limited by the descriptions presented herein, or by the descriptions presented in the accompanying documents.

We claim:

1. A therapeutic thermal device for application about an access site, comprising:
a semi-circumferential shaped housing having a top wall, a bottom wall, and a side wall, the top wall, the bottom wall, and the side wall defining a cavity;
a fluid tight bladder disposed within the cavity defined by the top wall, the bottom wall, and the side wall;
wherein the semi-circumferential shaped housing has an aperture formed in the semi-circumferential housing; and
wherein the semi-circumferential shaped housing comprises a straight edge portion along the circumference of the housing.

2. The therapeutic thermal device of claim 1, further comprising on the bottom wall of the housing at least one sterile peel-away paper exposing at least one adhesive point.

3. The therapeutic thermal device of claim 1, wherein the semi-circumferential housing comprises a polyvinyl chloride.

4. The therapeutic thermal device of claim 1, wherein the length of the straight edge portion is 25% of the circumference of the semi-circumferential shaped housing if the housing was a complete circle.

5. The therapeutic thermal device of claim 1, wherein the straight edge portion of the semi-circumferential shaped housing is comprised of three straight edges forming a three-sided substantially rectangular shape.

6. The therapeutic thermal device of claim 1, wherein the fluid tight bladder includes a solution and a separately maintained compartment for an organic or inorganic salt.

7. The therapeutic thermal device of claim 6, wherein the solution is water.

8. The therapeutic thermal device of claim 7, wherein the organic or inorganic salt is selected from the group consisting of ammonium nitrate, calcium ammonium nitrate, potassium nitrate, urea, and ammonium chloride.

9. The therapeutic thermal device of claim 1, wherein the device is placed about an access site exposing the access site for visual and physical examination while also allowing for indirect thermal therapy to the access site.

10. The therapeutic thermal device of claim 1, wherein the straight edge portion is placed at a bend or joint on a body located adjacent to an access site such as the location of the femoral, radial, brachial, pedal and popliteal arteries.

11. The therapeutic thermal device of claim 10, wherein the straight edge portions are tapered accordingly for comfortable and proper placement at a bend or joint on a body located adjacent to an access site such as the location of the femoral, radial, brachial, pedal and popliteal arteries.

12. The therapeutic thermal device of claim 1, wherein the lengths of the sides of the semi-circumferential shaped housing are equal.

13. A method for application of a therapeutic thermal device, comprising the steps of:
placing over an access site an aperture formed in a semi-circumferential housing having a top wall, a bottom wall, and a side wall, the top wall, the bottom wall, and the side wall defining a cavity; and
orienting a straight edge portion along the circumference of the semi-circumferential housing at a bend or joint on a body located adjacent to an access site such as the location of the femoral, radial, brachial, pedal and popliteal arteries.
wherein a fluid tight bladder disposed within the cavity defined by the top wall, the bottom wall, and the side wall includes a solution and a separately maintained compartment for an organic or inorganic salt to provide thermal therapy.
